(19) United States
(12) Patent Application Publication
(10) Pub. No.: US 2024/0142439 A1
(43) Pub. Date: May 2, 2024

(54) HYBRID IMMUNOASSAY DEVICES AND METHODS

(71) Applicant: Ortho-Clinical Diagnostics, Inc., Raritan, NJ (US)

(72) Inventor: Sydney Rachel SIMPSON, Rochester, NY (US)

(21) Appl. No.: 18/499,718

(22) Filed: Nov. 1, 2023

Related U.S. Application Data
(60) Provisional application No. 63/421,263, filed on Nov. 1, 2022.

(51) Int. Cl. G01N 33/53

(52) U.S. Cl. CPC G01N 33/5302

(57) ABSTRACT

Systems, methods, and devices are disclosed for a hybrid immunoassay process. An example method includes: applying an amount of a biological sample containing analytes to a first amount of detection molecules to form a mixture of labeled analytes and unlabeled analytes; causing the labeled analytes to bind to an amount of capture molecules, thus causing the mixture to comprise of bound labeled analytes and bound unlabeled analytes; applying an amount of wash fluid to a second amount of detection molecules; causing the second amount of detection molecules to dissolve into the wash fluid and form a detection molecule solution; applying the detection molecule solution to the mixture that includes the bound unlabeled analytes, thus forming additional bound labeled analytes as part of the mixture; and detecting, based on the bound labeled analytes, a concentration of analytes in the biological sample.

### CROSS-REFERENCE TO RELATED APPLICATIONS

The present application claims the benefit of U.S. Provisional patent application 63/421,263 filed Nov. 1, 2022, the entire contents of which are incorporated by reference herein.

### TECHNICAL FIELD

Certain aspects of the present disclosure generally relate to immunological devices and methods, and more specifically to immunoassay devices and methods for detecting analytes in biological samples.

### SUMMARY

The present disclosure presents new and innovative methods and compositions for devices, methods and systems for a hybrid immunoassay.

For example, in at least one embodiment, a device is disclosed for a hybrid immunoassay process. The device includes a wash zone configured to receive an amount of wash fluid; a pair of conjugate zones, comprising a first conjugate zone and a second conjugate zone, that branch from the wash zone and adjoin together at an adjoining point opposite from the wash zone; a capture channel zone interconnected to the adjoining point and storing an amount of capture molecules; and a removable barrier between the adjoining point and the second conjugate zone. Each conjugate zone may store an amount of detection molecules. For example, the first conjugate zone may store a first amount of detection molecules, and the second conjugate zone may store a second amount of detection molecules. The first conjugate zone may receive an amount of a biological sample comprising analytes and may cause the analytes to interact with the first amount of detection molecules. The removable barrier may be configured (e.g., in its on state) to initially divert a flow of a mixture comprising the amount of the biological sample and the first amount of detection molecules into the capture channel zone to interact with the amount of capture molecules. The wash zone, after receiving the amount of wash fluid, may cause the wash fluid to dissolve a second amount of detection molecules stored in the second conjugate zone, thus forming a detection molecule solution. The removable barrier may be configured (e.g., in its removed state) to cause the mixture comprising the amount of the biological sample and the first amount of detection molecules to interact with the detection molecule solution after interaction with the amount of capture molecules.

In some embodiments, the mixture comprising the amount of the biological sample and the first amount of detection molecules comprises: a first portion comprising analytes bound to detection molecules from the first amount of detection molecules, and a second portion comprising analytes that are bound to the detection molecules from the first amount of detection molecules.

In some embodiments, the first amount of detection molecules may comprise 0.1 to 100 nanograms of detection molecule, and the second amount of detection molecules may comprise 0.1 to 100 nanograms of detection molecule. The amount of capture molecules may comprise 0.1 to 500 nanograms of capture molecule. The amount of the biological sample containing the analytes may comprise 1 to 20 microliters of biological sample. The amount of wash fluid may comprise 1 to 15 microliters of wash fluid. The device may further comprise a permanent barrier configured to prevent a flow of the wash fluid between the wash zone and the first conjugate zone. Furthermore, a collection zone may be interconnected to the capture channel zone opposite of the adjoining point.

In a further embodiment, a method is provided for a hybrid immunoassay process. The method may include: applying an amount of a biological sample containing analytes to a first amount of detection molecules to form a mixture of labeled analytes and unlabeled analytes; causing the labeled analytes to bind to an amount of capture molecules, thus causing the mixture to comprise of bound labeled analytes and bound unlabeled analytes; applying an amount of wash fluid to a second amount of detection molecules; causing the second amount of detection molecules to dissolve into the wash fluid and form a detection molecule solution; applying the detection molecule solution to the mixture that includes the bound unlabeled analytes, thus forming additional bound labeled analytes as part of the mixture; and detecting, based on the bound labeled analytes, a concentration of analytes in the biological sample.

In some embodiments, the method may be performed via a branched micropillar device comprising a first conjugate zone and a second conjugate zone. The first amount of detection molecules and the second amount of detection molecules are stored in the first conjugate zone and the second conjugate zone, respectively. The amount of the biological sample containing the analytes may be applied to the first amount of detection molecules by placing the amount of the biological sample to the first conjugate zone. The branched micropillar device may further comprises a capture channel zone. Prior to causing the labeled analytes to bind to the amount of the capture molecules, the capture channel zone may store the amount of the capture molecules. In some embodiments, the first amount of detection molecules may comprise 0.1 to 100 nanograms of detection molecule, and the second amount of detection molecules may comprise 0.1 to 100 nanograms of detection molecule. The amount of capture molecules may comprise 0.1 to 500 nanograms of capture molecule. The amount of the biological sample containing the analytes may comprise 1 to 20 microliters of biological sample. The amount of wash fluid may comprise 1 to 15 microliters of wash fluid.

In a further embodiment, a system is disclosed for a hybrid immunoassay process. The system comprises: a branched device; an amount of wash fluid inserted into a wash zone of the branched device; a first amount of detection molecules stored in a first conjugate zone of the branched device and a second amount of detection molecules stored in a second conjugate zone of the branched device, wherein the first conjugate zone and the second conjugate zone comprise a pair of conjugate zones that branch from the wash zone, wherein the pair of conjugate zones adjoin together at an adjoining point opposite from the wash zone; an amount of a biological sample containing analytes, wherein the biological sample is inserted into the first conjugate zone; and an amount of capture molecules inserted into a capture channel zone. The capture channel zone may be interconnected to the adjoining point. The branched device causes the biological sample to interact with the first amount of detection molecules before interacting with the second amount of detection molecules. Furthermore, the branched device causes the biological sample to interact with the amount of capture molecules before interaction with a second amount of detection molecules. In some embodiments, the branched device is a branched micropillar device.

The features and advantages described herein are not all-inclusive and, in particular, many additional features and advantages will be apparent to one of ordinary skill in the art in view of the figures and description. Moreover, it should be noted that the language used in the specification has been principally selected for readability and instructional purposes, and not to limit the scope of the inventive subject matter.

### BRIEF DESCRIPTION OF THE FIGURES

FIG. 1 depicts an example one-step immunoassay device and process for the detection of analytes, according to an exemplary embodiment of the present disclosure.

FIG. 2 depicts an example two-step immunoassay device and process for the detection of analytes, according to an exemplary embodiment of the present disclosure.

FIG. 3 depicts an example hybrid immunoassay device and process for the detection and/or quantification of analytes, according to an exemplary embodiment of the present disclosure.

FIGS. 4A and 4B depicts a comparison of the analyte sensitivity of the one-step immunoassay device and process (FIG. 4A) with the analyte sensitivity of the hybrid immunoassay device and process (FIG. 4B), using a biological sample from a Hepatitis C positive patient, according to an exemplary embodiment of the present disclosure.

FIG. 5 depicts the analyte sensitivity of the two-step immunoassay device and process, using a biological sample from a syphilis positive patient, according to an exemplary embodiment of the present disclosure.

FIGS. 6A and 6B depicts a comparison of the analyte sensitivity of the one-step immunoassay device and process (FIG. 6A) with the analyte sensitivity of the hybrid immunoassay device and process (FIG. 6B), using the biological sample from the syphilis positive patient, according to an exemplary embodiment of the present disclosure.

### DETAILED DESCRIPTION

A clinical diagnostic assay (e.g., an immunoassay) may capture, detect, and/or quantify the presence or concentration of a macromolecule or a small molecule in a sample (e.g., a human sample) through the use of an antibody or an antigen. The macromolecule or small molecule detected by the clinical diagnostic assay may be referred to herein as an analyte. The sizes and types of analytes that can be captured, detected, and/or quantified by the clinical diagnostic assay can vary so long as proper antibodies and/or antigens having the required properties for the clinical diagnostic assay are developed.

In some clinical diagnostic assays, analytes are exposed to capture molecules, which comprise a unique binder specific for the analyte to hold the analyte in place. Furthermore, in some clinical diagnostic assays, analytes are exposed to detection molecules (e.g., labeling reagents). The detection molecules bind the analyte in order to allow the analyte to be detected. Mixing the analyte with the detection molecule before it is exposed to the capture molecule (referred to herein as a one-step immunoassay) allows for maximum detection at low analyte concentrations. However, the one-step immunoassay can result in a high dose hook phenomenon at higher analyte concentrations, wherein a false negative result is observed. Adding the detection antigen after the analyte has been captured, referred to herein as a two-step immunoassay process, prevents high-dose hook but can significantly lower the sensitivity or signal when low concentrations of analyte are present in some immunoassays.

Furthermore, conventional immunoassay devices and processes suffer from significantly lower analyte sensitivity and/or signal strength when low concentrations of analytes are present in a biological sample.

Various embodiments of the present disclosure address these shortcomings by providing methods, devices, and systems that can overcome high dose hook in high concentrations of analytes while retaining signal and sensitivity in the presence of low analyte concentrations. In at least one embodiment, an immunoassay process is disclosed where analytes (e.g., from a biological sample) are exposed to a first set of detection molecules before the analytes are permitted to be bound by capture molecules, forming captured analytes. Subsequently, the analytes are exposed to a second set of detection molecules by washing a solution containing the second set of detection molecules over the captured analytes. The process, referred to herein as a hybrid immunoassay process, allows for consistency in the capture, detection, and/or quantification of analytes for both high and low concentrations of analytes in a biological sample, thereby minimizing or eliminating the risk of false negative or false positive results.

In at least one embodiment, an immunoassay device is described that facilitates the hybrid immunoassay process. For example, the aforementioned immunoassay device, referred to herein as hybrid immunoassay device, may utilize a branched structure (e.g., a first conjugate zone and a second conjugate zone) to store and separate the two sets of detection molecules, and may utilize barriers (e.g., a permanent barrier and a removable barrier) to cause the analytes to be exposed to the first set of detection molecules before the analytes are permitted to be bound by capture molecules, and before the analytes are exposed to the second set of detection molecules. Furthermore, the hybrid immunoassay device may include a wash area allowing the washing of a solution containing the second set of detection molecules over the captured analytes, and may include a capture channel zone for storing the capture molecules.

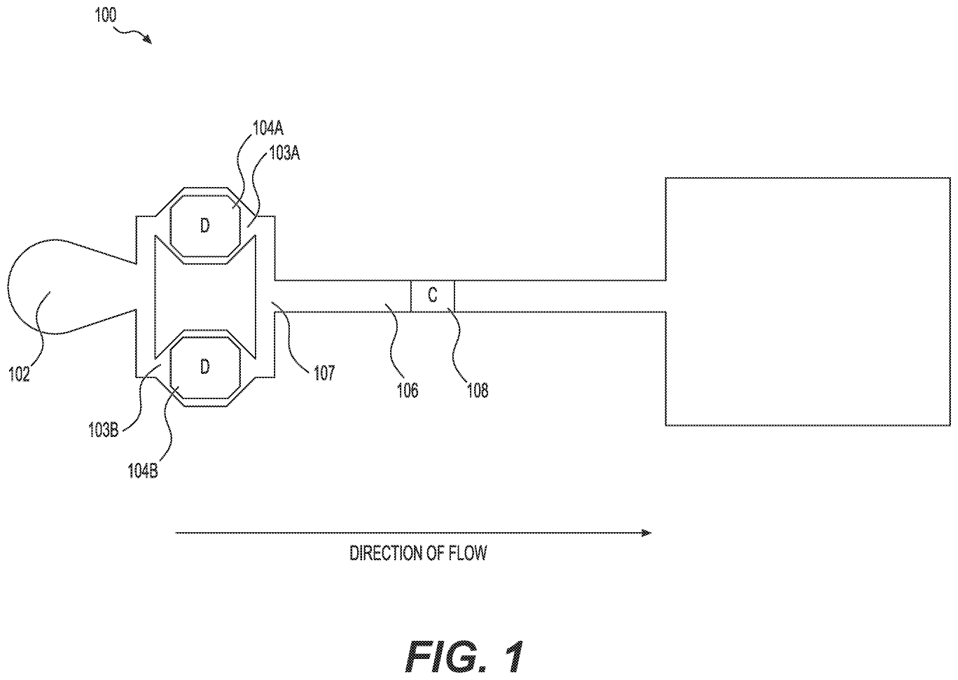

FIG. 1 is an illustration of an example one-step immunoassay device and process for the detection of analytes, according to an exemplary embodiment of the present disclosure. An example ‘one-step’ immunoassay device 100 may comprise a micropillar device. The one-step immunoassay device 100 may include an area where a biological sample comprising analytes (referred to herein as “analyte sample” or “sample”) is added (e.g., a sample zone 102), one or more areas where one or more amounts of detection molecules may be placed (e.g., in branched conjugate zones 103A-103B) and exposed to the sample, and an area for storing capture molecules 108 (e.g., capture channel zone 106). The detection molecules may comprise labeling reagents that may bind to the analyte, allowing the analyte to be detected. For example, a detection molecule may comprise an antibody or antigen receptor (e.g., if an analyte is an antigen) or a synthetic or naturally occurring antigen (e.g., if an analyte is an antibody). In some aspects, the detection molecule may include an illuminescent tag (e.g., a photoilluminescent tag) for easier detection. The capture molecules 108 may comprise a unique binder to the analyte to holds the analyte in place. In some aspects, the capture molecules 108 may hold an analyte bound to the detection molecule in place.

As shown in FIG. 1, the sample may be added to sample zone 102 that is a protruding part of the device at one end of the device. As will be discussed herein, this part of the device may be modified and/or repurposed for use in a hybrid immunoassay device and process. In some aspects, for example, as shown in FIG. 1, the amount of detection molecules may be divided and placed in separate and/or conjugate portions of the device. For example, the device may be branched (referred to herein as a branched device) and may comprise a first conjugate zone 103A and a second conjugate zone 103B, that stores a first amount 104A and a second amount 104B of the detection molecules, respectively. The first and second conjugate zones may lead to passageways that adjoin together at an adjoining point 107. The adjoining point 107 may be opposite from the above described protruding part of the device that, in a one-step immunoassay device, may be used as the sample zone 102. The adjoining point 107 may lead to or be connected to an area for storing capture molecules 108. Furthermore, the area for storing capture molecules 108 may be a narrow and elongated tube (e.g., as shown in capture channel zone 106) to ensure optimal exposure to the capture molecules 108.

In some embodiments, the immunoassay device 100 includes micropillars located within one or more of the sample zone 102, the first conjugate zone 103A, the second conjugate zone 103B, the adjoining point 107, and/or the capture channel zone 106. Micropillars may additionally or alternatively be located in an absorbing zone that is located at an end of the immunoassay device 100 that is located opposite from the sample zone 102. The micropillars are configured to improve the speed at which sample analytes flow through the immunoassay device 100.

The micropillars are described in PCT Publication No. WO 03/103835, which is incorporated herein by reference. The micropillars are projections or micro posts that protrude upwards from a surface of the immunoassay device 100. The spacing between the micropillars is such as to induce a capillary action in a liquid sample applied to the sample zone 102. In some embodiments, the micropillars may have the following dimensions: 69 μm in height, 46 μm in diameter, and placed at approximately 29 μm distance or distances from each other.

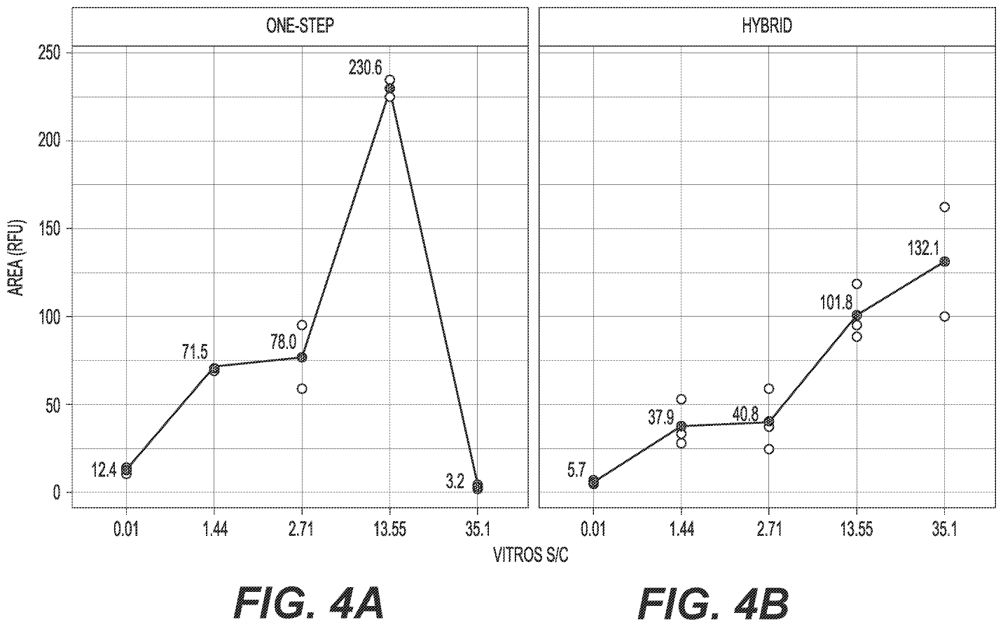

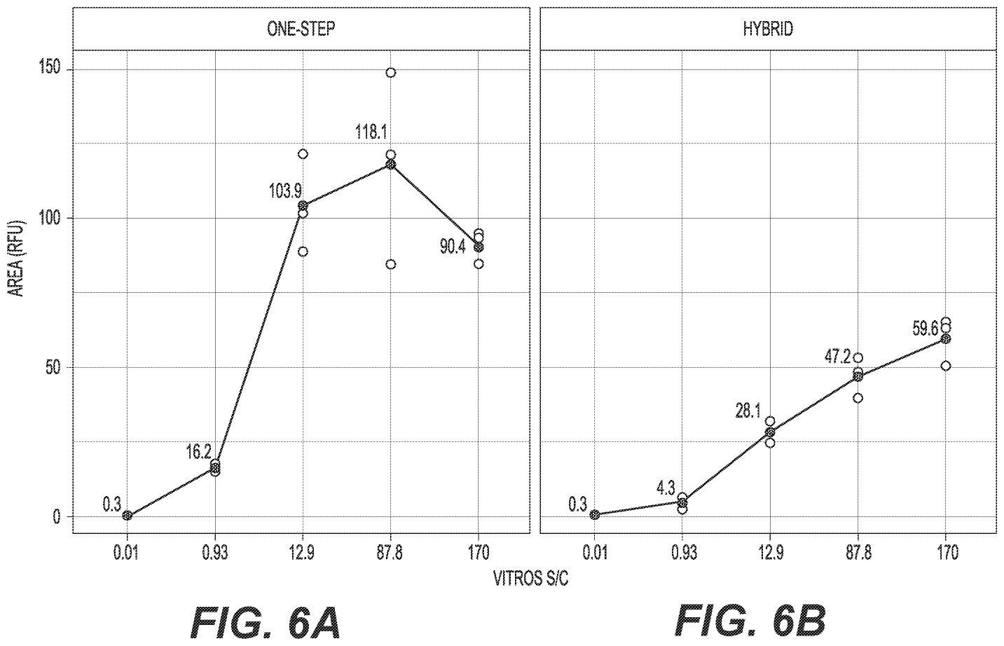

In an example one-step process, an amount of sample analytes may be added to the sample zone 102. The amount of analyte sample may flow down the device towards the one or more amounts of detection molecules. For example, as shown in FIG. 1, the amount of sample analytes may reach each of the conjugate zones, 103A and 103B, and may dissolve the amounts of detection molecules stored in both conjugate zones 103A and 103B. The resulting mixture may proceed towards the amount of capture molecules. For example, as shown in FIG. 1, the resulting mixture may flow into the capture channel zone 106, where analytes of interest from the resulting mixture (e.g., comprising a solution of detection molecules and sample analytes) may bind to capture molecules. By mixing the analyte sample with the detection molecules before exposing the resulting mixture to the capture molecules (e.g., for capture molecules to further bind to the analyte and/or to further bind to the analyte already bound to the detection molecule), the one-step immunoassay device and process may provide maximum detection and/or quantification for biological samples with low analyte concentrations. However, with higher analyte concentrations in the biological sample, the one-step immunoassay device and process may cause false negative detections and/or false quantifications of analytes in the sample (e.g., a high dose hook reading), as will be discussed in relation to FIGS. 4 and 6.

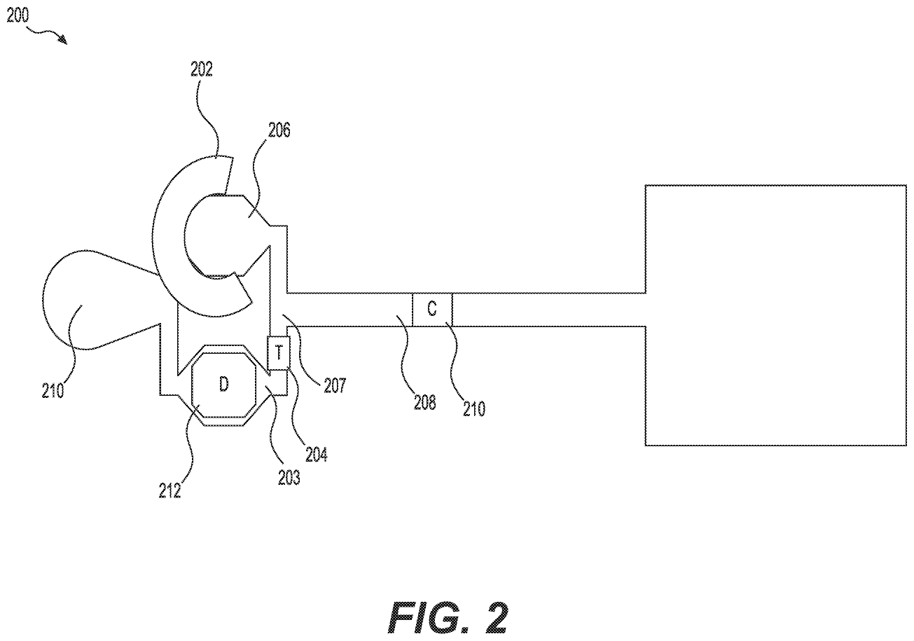

FIG. 2 is an illustration of an example two-step immunoassay device and process for the detection of analytes, according to an exemplary embodiment of the present disclosure. A ‘two-step’ immunoassay device 200 may comprise a device 200. Thus, like the one-step immunoassay device 100, the two-step immunoassay device may comprise a protruding end 210, branched conjugate zones 206 and 203, an adjoining point 207, and a channel capture zone 208. However, unlike the device 100 used for the one-step immunoassay process in FIG. 1, the device 200 used in the two-step immunoassay process may include a barrier 202. The barrier 202 (also referred to herein as “permanent barrier” 202) may permanently and/or significantly impede the flow of solutions, substances, and/or mixtures described in the two-step immunoassay process. As shown in FIG. 2, the permanent barrier 202 may be placed behind one of the conjugate zones of the immunoassay device 200 (e.g., conjugate zone 206). Moreover, the conjugate zone 206 (referred to herein as the “first conjugate zone”) may not store any amount of detection molecules. Furthermore, the device 200 used in the two-step immunoassay process may include a second barrier 204. The second barrier 204, referred to herein as removable barrier 204, may be removable, and may be placed in front of the other conjugate zone (e.g., conjugate zone 203, as shown in FIG. 2, referred to herein as “second conjugate zone”). In some aspects, the removable barrier 204 may include an “on” state and a “removed” state, so as to prevent the flow of solutions, substances, and/or mixtures in the “on” state, while allowing the flow of solutions, substances, and/or mixtures in the “removed” state. For example, the removable barrier may comprise a valve that closes during the “on” state obstructing a passageway between the second conjugate zone 203 and an adjoining point between the first conjugate zone, the second conjugate zone, and the capture channel zone, as shown in FIG. 2. The valve may be controlled manually (e.g., by physically removing the removable barrier 206 to commence the “removed” state) and/or may be triggered automatically based on events associated with the immunoassay process, as will be discussed herein.

In an example embodiment of the two-step immunoassay process, a sample of analytes (e.g., a biological sample) may be added to the first conjugate zone 206. As previously described, the first conjugate zone 206 may be empty or may otherwise not store any amount of detection molecules. The sample containing the analytes may flow into the capture channel zone 208 (e.g., via the adjoining point 207) where analytes of interest may be bound by capture molecules 210 stored in the channel capture zone 208. Moreover, the “on” state of the removable barrier 204 may prevent the sample from flowing into the second conjugate zone 203, and may thus cause the sample the flow directly into the channel capture zone 208 instead. The two-step immunoassay process may further include adding a wash fluid to the protruding end 210 of the two-step immunoassay device 200. The protruding end 210, which may be modified and/or configured to receive the wash fluid, may be referred to herein (e.g., for the two-step immunoassay process and the hybrid immunoassay process) as a wash zone. The second conjugate zone 203 may store an amount of detection molecules. The removable barrier 204 may then switch to a removed state (e.g., by causing the removal of the removable barrier 204). The wash fluid may flow into the second conjugate zone 203, mixing and/or dissolving the amount of detection molecules 212 stored in the second conjugate zone 203. The resulting mixture may then flow towards the adjoining point 207 (since the removal barrier is removed) and may then proceed into the capture channel zone 208. At the capture channel zone 208, the resulting mixture comprising the dissolved amount of detection molecules may wash over the bound analytes of interest in the capture channel zone 208. The interaction between the dissolved detection molecules with the analytes bound to the capture molecules may cause only the bound analytes to be labeled. The analytes of interest from the sample that did not bind to capture molecules 210 may not get exposed to detection molecules. Thus, the two-step immunoassay process may mitigate the risk of high-dose hook (e.g., false negative detection and/or quantification of analytes at high analyte concentrations). However, since the detection molecules may only be able to bind to analytes that are already bound to the capture molecules, the two-step process may significantly lower analyte sensitivity or signal strength when there are low concentrations of analyte in a sample.

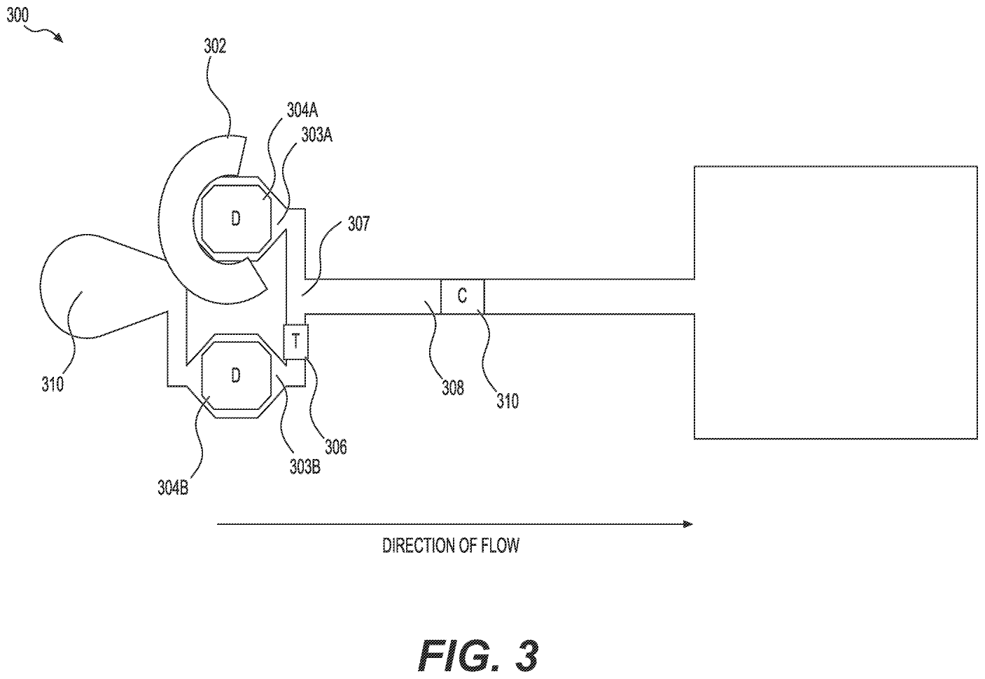

FIG. 3 is an illustration of an example hybrid immunoassay device and process for the detection and/or quantification of analytes, according to an exemplary embodiment of the present disclosure. A hybrid immunoassay device 300 may comprise a branched micropillar device 300. However, like the two-step immunoassay device 200, the hybrid immunoassay device 300 may include: a protruding end 310; conjugate zones 303A and 303B branching from the protruding end 210 and adjoining together at an adjoining point 207; a channel capture zone 208; a permanent barrier 302 permanently and/or significantly impeding the flow of solutions, substances, and/or mixtures directly between the first conjugate zone 303A and the protruding end 310; and a removable barrier 306 between the second conjugate zone 303B and the adjoining point 307. Like the removable barrier 204 of the two-step immunoassay device 200, the removable barrier 306 of the hybrid immunoassay device 300 may, during an “on” state, obstruct the flow of solutions, substances, and/or mixtures directly between the second conjugate zone 303B and the adjoining point 307, but may, during a “removed” state, allow the flow of solutions, substances, and/or mixtures. For example, the removable barrier may comprise a valve that may be controlled manually (e.g., by physically removing the removable barrier 306 to commence the “removed” state) and/or may be triggered automatically based on events associated with the hybrid immunoassay process, as will be discussed herein.

However, like the one-step immunoassay device 100 described in relation to FIG. 1, the hybrid immunoassay device 300 may store, within its first conjugate zone 303A and its second conjugate zone 303B, a first amount 304A and a second amount 304B of detection molecules, respectively, and may store, within the capture channel zone 308, an amount of capture molecules 310.

In at least one embodiment, a hybrid immunoassay process may begin with adding a sample (e.g., a biological sample) containing a plurality of analytes directly to the first conjugate zone 303A. As the first conjugate zone 303A already stores a first amount of detection molecules 304A, the placement of the sample may cause the analytes to come into direct contact with the first amount of detection molecules 304A. The direct contact may result in a mixture of labeled analyte (e.g., analytes bound to the detection molecules) and unlabeled analyte (e.g., analytes from the sample that are not bound to the detection molecules). The mixture may then flow toward the capture channel zone 308 (e.g., via the adjoining point 307). Furthermore, the removable barrier 306 may prevent the mixture from flowing into the second conjugate zone 303B, thus causing the mixture to be diverted to the capture channel zone 308. At the capture channel zone 308, analytes of interest from the mixture may be bound to capture molecules from the amount of capture molecules 310. The analytes of interest that bind to the capture molecules may include both labeled and unlabeled analytes (e.g., analytes previously bound to and not bound to detection molecules, respectively). However, in some embodiments, capture molecules may be selected and/or synthesized to only bind to the labeled analytes.

The hybrid immunoassay process may further include adding an amount of wash fluid into the protruding end 310 of the hybrid immunoassay device 300. The protruding end 310, which may be modified and/or configured to receive the wash fluid, may be referred to as a wash zone. The hybrid immunoassay process may further include causing the removable barrier to be in a “removed” state (e.g., by physically removing the removable barrier 306 or by automatically switching the removable barrier 306 from the previously “on” state to the “removed” state). The removed state may cause the wash fluid to flow into the second conjugate zone 304B. Also or alternatively, at least some of the wash fluid may flow into the second conjugate zone 304B even before the removable barrier 306 switches to the removed state.

The second amount of detection molecules 304B stored in the second conjugate zone 303B may dissolve into the wash fluid, thus resulting in a wash fluid solution containing the second amount of detection molecules (referred to herein as detection molecule solution). When the removable barrier is in a removed state, the detection molecule solution may flow into the capture channel zone 308 (e.g., via the adjoining point 307). At the capture channel zone 308, the detection molecule solution may come in contact with the previously described mixture that includes the analytes (e.g., labeled and/or unlabeled analytes) bound to the capture molecules. The detection molecule solution may wash over the bound analytes of interest in the capture channel zone 308, thus allowing any unlabeled analytes that have not yet been bound to (e.g., labeled by) the first amount of detection molecules 304A to bind to detection molecules now in the detection molecule solution (e.g., originating from the second amount of detection molecules 304B). By allowing any unlabeled analytes from the sample to have a “second chance” at getting bound by the detection molecules (e.g., after the sample has been exposed to and bound by capture molecules), the hybrid immunoassay process and device thus mitigates the high-dose hook effect of the one-step immunoassay process and device. Furthermore, by allowing analytes from the sample to bind to detection molecules (e.g., from the first amount of detection molecules 304A) before binding to the capture molecules, the hybrid immunoassay process and device can maintain analyte sensitivity and signal strength even when there are low concentrations of analyte in the sample.

In some embodiments, the amount of the sample containing the analytes that is added to the first conjugate zone 303A may range from 1 to 20 microliters (e.g., 1.5 to 3.5 microliters). This range may be optimal and/or significant for the disclosed hybrid immunoassay device and process because increasing the amount of sample increases the total amount of detectable analyte. Furthermore, the first and second amounts of detection molecules, 304A and 304B, respectively, may range from 0.1 to 100 nanograms (e.g., 1 to 16 nanograms). This range may be optimal and/or significant for the disclosed hybrid immunoassay device and process because there must be enough detection molecules to label analyte but not enough to oversaturate and inhibit analyte capture by capture molecules. The amount of the capture molecules stored in the capture channel zone 308 may range from 0.1 to 500 nanograms (e.g., 4 to 50 nanograms). This range may be optimal and/or significant for the disclosed hybrid immunoassay device and process because capture molecules must be accessible and present in high enough concentrations to capture analyte, if present, at detectable levels without preventing binding of the second conjugate to captured analyte which did not bind the first conjugate. Furthermore, the amount of the wash fluid used in the hybrid immunoassay device and process may range from 1 microliter to 15 microliters (e.g., 3 to 10 microliters). This range may be optimal and/or significant for the disclosed hybrid immunoassay device and process because enough wash must be present to both dissolve the conjugate in the second conjugate zone, and wash remaining unbound detection molecules from the microfluidic channel.

FIGS. 4A and 4B depicts a comparison of the analyte detection and quantification by the one-step immunoassay device and process (FIG. 4A) with the analyte detection and quantification by the hybrid immunoassay device and process (FIG. 4B), using a biological sample from a Hepatitis C Virus (HCV)-positive patient, according to an exemplary embodiment of the present disclosure. FIG. 4A shows the analyte detection and quantification (e.g., measured in areas of relative fluorescence units (RFUs)) for various concentration of analytes in a biological sample of the HCV-positive patient, as used in a one-step immunoassay device and process (e.g., as described in relation to FIG. 1). Furthermore, FIG. 4B shows a detection and a quantification (e.g., the amount in a sample) of analytes (e.g., measured in areas of relative fluorescence units (RFUs)) for various concentration of analytes in the biological sample of the HCV-positive patient, as used in a hybrid immunoassay device and process (e.g., as described in relation to FIG. 3). For each graph, the X-axis shows a VITROS® MicroWell Hepatitis C Virus assay signal to cutoff for the samples and is used as a reference. The Y-axis shows the signal in relative fluorescent units (RFU) that was detected by an LRE reader. In these examples, the different concentrations were obtained by diluting plasma from the HCV-positive patient at different concentrations of HCV-negative plasma. For each graph, the plotted signals of individual replicates (e.g., as shown by the white dots) were averaged (e.g., as shown by the black dots), resulting in a best-fit line. Furthermore, the capture and detection molecules used in these examples are HCV antigens and the analytes of interest are antibodies against these antigens. As shown in FIG. 4A, the one-step immunoassay device and process results in a characteristic high-dose hook effect (e.g., false negative signal for the presence of analytes) at high concentrations. The high dose hook effect thus paints an inaccurate reading (e.g., based on RFU) of the actual concentration of analytes (e.g., as measured by RFUs) at high concentrations of the analyte. In contrast, FIG. 4B, corresponding to the hybrid immunoassay device and process, does not show the high dose hook effect at high concentrations. As shown in FIG. 4B, the quantification of analytes (e.g., as indicated by RFUs) continues to rise with increasing concentrations of the analyte in the sample.

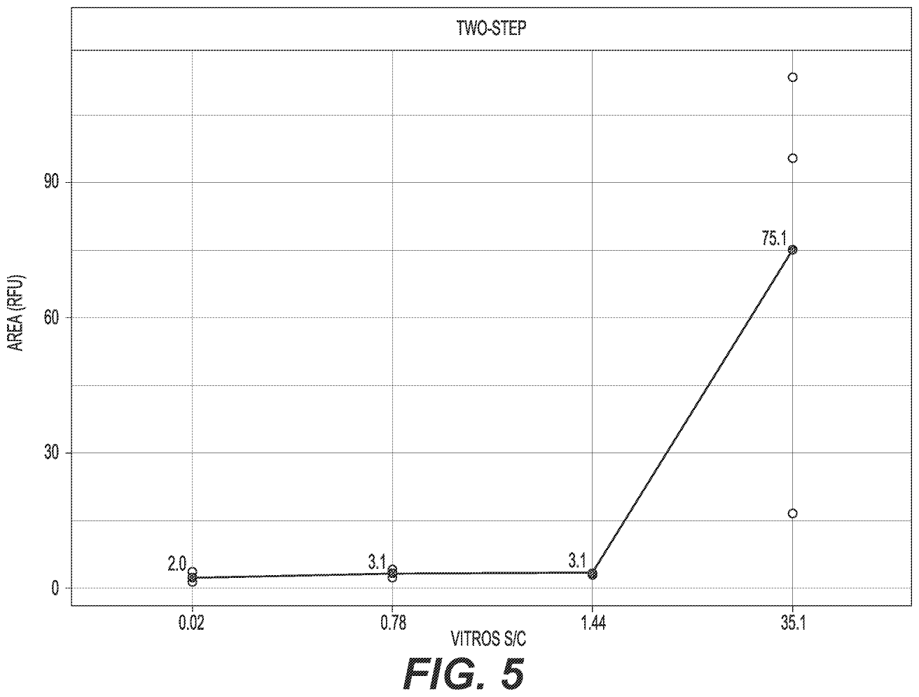

FIG. 5 is a graph showing the analyte detection and quantification by the two-step immunoassay device and process, using a biological sample from a syphilis-positive patient, according to an exemplary embodiment of the present disclosure. The different concentrations of analytes were obtained by diluting plasma from the syphilis-positive patients with syphilis-negative plasma, and used in the two-step immunoassay device and process. While the X-axis shows the various concentration of analytes in the biological sample of the syphilis-positive patient, the Y-axis shows a quantification of analytes (e.g., the amount of analytes in a sample) detected by the two-step immunoassay device (e.g., as measured in areas of relative fluorescence units (RFUs)). As shown in FIG. 5, the two-step immunoassay process mitigates above identified problem of one-step immunoassay devices and processes, where a high dose hook (e.g., a false negative amount of analytes) is inaccurately presented at at higher concentrations of analytes. To the contrary, FIG. 5 does not show any high dose hook at high analyte concentrations. However, there is a diminished analyte sensitivity at lower concentrations of the analyte. For example, at concentrations of 0.02, 0.78, and 1.44 sample to control index ratios (S/C), the two-step immunoassay registers a fairly flat quantification of analyte levels in the sample, as measured by RFUs, even though the concentrations are rising. As previously discussed in relation to FIG. 2, analytes are exposed to detection molecules only after being exposed to capture molecules in a two-step immunoassay process. This process may cause only the analytes that are bound to the capture molecules to bind to and get labeled by the detection molecules, thus causing low sensitivity and low signal strength at low analyte concentrations.

FIGS. 6A and 6B is a comparison of the analyte detection and quantification by the one-step immunoassay device and process (FIG. 6A) with the analyte detection and quantification by the hybrid immunoassay device and process (FIG. 6B), using the biological sample from the syphilis-positive patient, according to an exemplary embodiment of the present disclosure. Specifically, FIG. 6A shows the analyte detection and quantification by the one-step immunoassay device and process, and graph FIG. 6B shows the analyte detection and quantification by the hybrid immunoassay device and process. As expected, and previously described in relation to FIG. 1, the one-step immunoassay device and process characteristically causes a high-dose hook effect, where there is a false negative detection and quantification of analytes at greater analyte concentrations in the sample. For example, at an analyte concentration of 170 S/C, the one-step immunoassay device and process appears to indicate a decline in analytes (e.g., from 118.0 to 90.4 RFUs) even though there is an increase in concentrations. However, the hybrid immunoassay device and process, as shown in FIG. 6B, does not exhibit the high dose hook phenomenon at these concentrations (e.g., at or near 170 S/C). Instead, the detection and quantification of analytes in the sample by the hybrid immunoassay device and processes rises commensurately with the increase in analyte concentration. Furthermore, the hybrid immunoassay device and process maintains sensitivity to analytes in the sample even at low concentrations (e.g., 0.3 RFUs at 0.01 S/C, and 4.3 RFUs at 0.93). Thus, the hybrid immunoassay device and process mitigate the issue of low sensitivity for low analyte concentrations, which is characteristic of the two-step immunoassay device and process. Thus, the experimental data, as shown and described in relation to FIGS. 4-6, demonstrate that the hybrid immunoassay device and process is a novel and nonobvious approach to improving assay sensitivity (e.g., at low analyte concentrations in a sample) while eliminating high dose hook effect (e.g., at high analyte concentrations in the sample).

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A device comprising:
a wash zone configured to receive an amount of wash fluid;
a pair of conjugate zones comprising a first conjugate zone and a second conjugate zone, wherein the pair of conjugate zones branch from the wash zone and adjoin together at an adjoining point opposite from the wash zone, wherein the first conjugate zone stores a first amount of detection molecules, wherein the second conjugate zone stores a second amount of detection molecules, wherein the first conjugate zone receives an amount of a biological sample comprising analytes and causes the analytes to interact with the first amount of detection molecules;
a capture channel zone interconnected to the adjoining point and storing an amount of capture molecules; and
a removable barrier between the adjoining point and the second conjugate zone, wherein the removable barrier is configured to initially divert a flow of a mixture comprising the amount of the biological sample and the first amount of detection molecules into the capture channel zone to interact with the amount of capture molecules;
wherein the wash zone, after receiving the amount of wash fluid, causes the wash fluid to dissolve a second amount of detection molecules stored in the second conjugate zone, thus forming a detection molecule solution, wherein the removable barrier is configured to cause the mixture comprising the amount of the biological sample and the first amount of detection molecules to interact with the detection molecule solution after interaction with the amount of capture molecules.

2. The device of claim 1, wherein the mixture comprising the amount of the biological sample and the first amount of detection molecules comprises:
a first portion comprising analytes bound to detection molecules from the first amount of detection molecules, and
a second portion comprising analytes that are bound to the detection molecules from the first amount of detection molecules.

3. The device of claim 1, wherein the device further comprises:
a permanent barrier configured to prevent a flow of the wash fluid between the wash zone and the first conjugate zone.

4. The device of claim 1, wherein the removable barrier is at an on state prior to a removed state;
wherein the on state of the removable barrier is configured to cause the biological sample to interact with the first amount of detection molecules before the interaction with the second amount of detection molecules; and
wherein the removed state of the removable barrier is configured to cause the biological sample to interact with the detection molecule solution formed from the second amount of detection molecules dissolving into the wash fluid.

5. The device of claim 1, further comprising:
a collection zone interconnected to the capture channel zone opposite of the adjoining point.

6. The device of claim 1, wherein the first amount of detection molecules and the second amount of detection molecules are each independently about 0.1-100 ng.

7. The device of claim 1, wherein the amount of capture molecules is about 0.1-500 ng.

8. The device of claim 1, wherein the amount of the biological sample containing the analytes is about 1-20 microliters.

9. A method comprising:
applying an amount of a biological sample containing analytes to a first amount of detection molecules to form a mixture of labeled analytes and unlabeled analytes;
causing the labeled analytes to bind to an amount of capture molecules, thus causing the mixture to comprise of bound labeled analytes and bound unlabeled analytes;
applying an amount of wash fluid to a second amount of detection molecules;
causing the second amount of detection molecules to dissolve into the wash fluid and form a detection molecule solution;
applying the detection molecule solution to the mixture that includes the bound unlabeled analytes, thus forming additional bound labeled analytes as part of the mixture;
detecting, based on the bound labeled analytes, a concentration of analytes in the biological sample.

10. The method of claim 9, wherein the first amount of detection molecules and the second amount of detection molecules are each independently about 0.1-100 ng.

11. The method of claim 9, wherein the amount of capture molecules is about 0.1-500 ng.

12. The method of claim 9, wherein the amount of the biological sample containing analytes is about 1-20 microliters.

13. The method of claim 9, wherein the amount of wash fluid applied is about 1-15 microliters.

14. The method of claim 9, wherein the method is performed via a branched micropillar device,
wherein the micropillar device comprises a first conjugate zone and a second conjugate zone,
wherein the first amount of detection molecules and the second amount of detection molecules are stored in the first conjugate zone and the second conjugate zone, respectively, and
wherein the amount of the biological sample containing the analytes is applied to the first amount of detection molecules by placing the amount of the biological sample to the first conjugate zone.

15. The method of claim 14, wherein the branched micropillar device further comprises a capture channel zone,
wherein, prior to causing the labeled analyte to bind to the amount of the capture molecules, the capture channel zone stores the amount of the capture molecules.

16. The method of claim 15, wherein the branched micropillar device further comprises a wash zone,
wherein the amount of the wash fluid is applied to the second amount of detection molecules by placing the amount of the wash fluid in the wash zone
wherein a permanent barrier separates the first conjugate zone and the wash zone,
wherein the permanent barrier prevents a direct flow of the wash fluid between the wash zone and the first conjugate zone.

17. The method of claim 16, wherein the detection molecule solution is applied to the mixture after causing the labeled analyte to bind to the amount of capture molecules and forming the mixture comprising the bound labeled analytes and the bound unlabeled analytes.

18. The method of claim 17, wherein a removable barrier separates the second conjugate zone from the capture channel zone,
wherein the detection molecule solution is applied to the mixture after causing the labeled analyte to bind to the amount of capture molecules by removing the removable barrier.

19. A system comprising:
a branched device;
an amount of wash fluid inserted into a wash zone of the branched device;
a first amount of detect molecules stored in a first conjugate zone of the branched device and a second amount of detection molecules stored in a second conjugate zone of the branched device, wherein the first conjugate zone and the second conjugate zone comprise a pair of conjugate zones that branch from the wash zone, wherein the pair of conjugate zones adjoin together at an adjoining point opposite from the wash zone;
an amount of a biological sample containing analytes, wherein the biological sample is inserted into the first conjugate zone; and
an amount of capture molecules inserted into a capture channel zone, wherein the capture channel zone is interconnected to the adjoining point;
wherein the branched device causes the biological sample to interact with the first amount of detection molecules before interacting with the second amount of detection molecules, and
wherein the branched device causes the biological sample to interact with the amount of capture molecules before interaction with a second amount of detection molecules.

20. The system of claim 19, wherein the branched device is a branched micropillar device.
